(19) United States
(12) Patent Application Publication
(10) Pub. No.: US 2003/0105456 A1
(43) Pub. Date: Jun. 5, 2003

(54) Apparatus and methods for prevention of age-related macular degeneration and other eye diseases

(76) Inventor: J.T. Lin, Oviedo, FL (US)

Correspondence Address: J.T. LIN, 4532 Old Carriage Trail, Oviedo, FL, 32765, US

(21) Appl. No.: 10/000,163

(22) Filed: Dec. 4, 2001

(51) Int. Cl. A61B 18/18

(52) U.S. Cl. 606/5; 606/3; 606/10

(57) ABSTRACT

Surgical apparatus and surgical methods are proposed for the prevention of age-related macular degeneration (AMD) and choroidal neovascularization (CNV), and other eye diseases such as glaucoma by removal of the sclera tissue to reduce its rigidity and increase the flood flow and decrease pressure in the choriocapillaris. The disclosed preferred embodiments of the system consists of a tissue ablation means and a control means of ablation patterns and a fiber delivery unit. The basic laser beam includes UV lasers and infrared lasers having wavelength ranges of (0.15-0.36) microns and (0.5-3.2) microns and diode lasers of about 0.98, 1.5 and 1.9 microns. AMD and CNV are prevented, delayed or reversed by using an ablative laser to ablate the sclera tissue in a predetermined patterns outside the limbus to increase the elasticity of the sclera tissue surrounding the eye globe The surgery apparatus also includes non-laser device of radio frequency wave, electrode device, bipolar device and plasma assisted device

### BACKGROUND OF THE INVENTION

1. Field of the Invention

The present invention relates to methods and apparatus for the prevention of age-related macular degeneration and other eye diseases.

2. Background and Prior Art

Age-related macular degeneration (AMD) is the leading cause of central visual loss in patients older than 50 years of age in the United States. The 10% of patients with wet degeneration accounts for 90% of the patients with severe vision loss to 20/200 or worse. The majority of eyes suffer severe visual loss of a result of Choroidal neovascularization (CNV), which is the formation of new blood vessels either between the retinal pigment epithelium and Bruch membrance or the subretinal space.

CNV is a common manifestation of a variety of macular diseases and can result in severe vision loss. Typically, CNV complicates AMD, but it also can be seen in pathologic myopia, ocular histoplasmosis, angioid streaks, and ocular inflammatory diseases, and as an idiopathic condition. See Atlas of Ophthalmic Surgery, Chapt. 8, ed. by N. Jaffe, (Mosby-Wolfe, 1996).

Recently, there has been an explosion of treatment options, including the use of photodynamic therapy with verteporfin, radiation, transpupillary thermotherapy, and feeder vessel photo-coagulation and surgical techniques such as submacular surgery and macular translocation. See J. Paerlman et al, Contemporary Opthalmology, November 2001 (Lippincott Williams & Wilkins, MD).

The proven effective treatment for AMD is laser photocoagulation (LPD). However, it was reported that treatment of well-defined subfoveal CNV was beneficial, but most patients experienced an immediate decline in vision because of damage to the overlying neurosensory retina. Freund et al demonstrated that only 13% of patients with CNV from AMD are eligible for treatment by LPD. (See Freund K B, Am. J Ophthalmol 1993; vol. 115, pp. 786-91). Therefore “prevention” of AMD is more important than cure it. The present invention shall proposed methods for the prevention, delaying or reversal of AMD. Before introducing these methods, we shall review the background of the risk factors causing AMD as follows.

The pathogenesis of AMD is not entirely known, but defects in Bruch's membrane are associated with at least some forms of CNV and are seen histologically in cases for which clinical-pathologic correlation is available. The presence of the abnormal vessels, combined with the development of subretinal transudates and hemorrhage, ultimately results in irreparable damage to the overlying neurosensory retina and permanent loss of vision.

The vascular model proposed by E. Friedman (Am J Ophthalmol vol. 130, pp. 658-663 2000) stated that AMD is the result of the accumulation of lipid in the sclera and in Bruch Membrance, progressively increasing the stiffness of these tissues and increasing the postcapillary resistance of the choroidal vasculature, situated between the progressively noncompliant sclera and noncompressable contents of the globe. This model also stated that in addition to decreasing choroidal blood flow, the increase in resistance or elevation the hydrostatic pressure of the choriocapillaris, enhancing leakage and deposition of extracellular proteins and lipids. In AMD, the location of the lipid deposition is also a function of the intravascular hydrostatic pressure. The lipids deposited in the sclera may originate in scleral vessels or they may reach the sclera from the choroids by diffusion or filtration down the transsclera hydrostatic pressure gradient.

In addition to the above risk factors of AMD and CNV, it was also reported that hyperopia is frequently identified as a risk factor for AMD in large case-control epidemiological studies. (See Tang et al, German J Opthalmol 1993, vol. 2, pp.10-13). The vascular model of Freidman suggested that this can be attributed to the increased scleral rigidity associated with hyperopia. The present inventor proposes that scleral rigidity should cause “presbyopia” rather than “hyperopia”. In Lin's U.S. Pat. No. 6,258,082, the present inventor proposed that presbyopia may be reversed by a laser treatment which increases the elasticity of the sclear-ciliary-zonule complex, where presbyopic patients are treated by increasing their near vision accommodation. In the present invention, we propose to use the mechanism based on an “elastic theory” for the new application of prevention, delay or reversal of AMD (or CNV) by reducing their risk factors which includes choriodal low blood flow and the choriocapillaris high pressure.

We proposed that the laser ablated sclera tissue “gap” may be filled in by the sub-conjunctival tissue within few days after the surgery. This filled in sub-conjunctival tissue is much more elastic than the original sclera tissue and therefore cause the scleral tissue surrounding the eye globe to become more elastic or less rigid. This “elastic” mechanism shall then lower or eliminate the risks factors causing AMD, namely the low blood flow in the choriod and high hydrostatic pressure. Formation of lipids, drusen and neovascularization localized in the periphery of the fundus may be prevented, reduced or even reversed after the scleral tissue surrounding the globe becomes more elastic achieved by methods proposed in the present invention.

Therefore, one objective of the present invention is to provide an apparatus and method to lower or eliminate the risks factors causing AMD.

It is yet another objective of the present invention to use a laser system with scanning device or fiber-coupled delivery unit to control the ablation patterns, location, size and shapes on the sciera tissue.

It is yet another objective of the present invention to define the non-thermal lasers for efficient tissue ablation.

It is yet another objective of the present invention to define the optimal laser parameters and the ablation patterns for best clinical outcome for preventing AMD with minimum side effects.

It is yet another objective of the present invention to provide a new mechanism which supports the projected clinical outcome of AMD prevention and the efficacy and long term efficacy of this procedure. The mechanism presented in the present patent is to remove portion of the sclera tissue which is filled in by sub-conjunctiva tissue to increase the flexibility of the scleral area and in turn lower the risk factors of vessel stiffening causing AMD.

It is yet another objective of the present invention to use a non-laser method to remove portion of the scleral tissue and achieve the similar clinical outcome as that of laser methods, as far as this removed scleral area can be filled in by the sub-conjunctival tissue. These non-laser methods shall include, but not limited to, physical blades or knife, electromagnetic wave such as radio frequency wave, electrode device, bipolar device and plasma assisted electrosurgical device.

The present invention described in great detail for the prevention of AMD may be extended to other eye diseases including glaucoma which require lowering of the intraocular pressure (IOP). For the case of glaucoma, the laser and non-laser devices may be used to remove sclera tissue in the area where Schlemm's channel is located followed by a removal of a small portion of the iris underlying this area Based on the elastic theory, the reduce of rigidity of the sclera shall also IOP to prevent, delay or treat glaucoma.

The invention having now been fully described, it should be understood that it may be embodied in other specific forms or variations without departing from the spirit or essential characteristics of the present invention. Accordingly, the embodiments described herein are to be considered to be illustrative and not restrictive.

### SUMMARY OF THE INVENTION

The preferred embodiments of the basic surgical lasers of the present invention shall include (a) infrared (IR) lasers having wavelengths range of about (1.4-3.2) microns including but not limited to solid state lasers of Er:glass, Ho:YAG, Er:YAG, Er:YSGG, infrared gas lasers, solid-state lasers converted by optical parametric oscillation (OPO); (b) ultraviolet (UV) lasers having wavelength range of about (190-355) nm, such as ArF (at 193 nm) and XeCl (at 308 nm) excimer lasers, nitrogen laser (at 337 nm) and harmonics of solid-state lasers using frequency up-conversions; (c) semiconductor diode lasers at about 980 nm, (1.3-1.55) microns, and (1.8-2.1) microns; (d) flash-lamp-pumped and diode-pumped solid state lasers having wavelength range of about (190-355) nm and (2.7-3 2) microns such as Er:YSGG, Er:YAG, Nd:YAG, Er:glass and Ti:saphire laser and their harmonic generation; (e) short pulse infrared lasers at (1.0-1.4) microns, with pulse duration of between about 1.0 femtosecond and 10 nanoseconds.

It is yet another preferred embodiment is to couple the basic lasers by a fiber and deliver the laser beam to the treated area of the eye by a hand held piece which is further connected to a fiber-tip at various shapes.

It is yet another preferred embodiment to focus the laser beams into a desired spot size on the treated area of the eye. Various ablation patterns may be generated manually via the fiber-connected hand piece including multiple dotted rings and radial line excisions outside the limbus.

It is yet another preferred embodiment to focus the laser beams into a means of scanning device such that various ablation patterns may be generated by controlling the scanning device. The scanning devices shall include the use of a motorized reflection mirror, refractive optics device or manually controlled translation device.

It is yet another preferred embodiment is to remove, by any methods either laser or non-laser, portion of the sclera tissue which is filled in by sub-conjunctiva tissue to increase the flexibility of the scleral area and in turn reduce the risk factors of AMD and CNV.

The preferred embodiment for non-laser methods shall include, but not limited to, physical blades or knife, electromagnetic wave such as radio frequency wave, electrode device, bipolar device and plasma assisted electrode device.

It is yet another preferred embodiment to open the conjunctiva layer prior to the laser ablation of the under-layer of the sciera tissue for a better control of the ablation depth and for safety reasons. It is yet another preferred embodiment is that the conjunctiva layer may be lifted to generate the “gap” for fiber tip to insert into the gap and ablate the desired patterns underneath and to avoid or minimize bleeding or infection.

Further preferred embodiments of the present invention will become apparent from the description of the invention which follows.

### BRIEF DESCRIPTION OF THE DRAWINGS

FIG. 1 is a schematics showing retinal development of abnormal blood vessels (drusen) in the subretinal space which will cause CNV.

FIG. 2 shows various layers of eye tissue outside limbus, where the scleral tissue ablated by a laser is filled in by sub-conjunctival (episclera) tissue after the conjunctiva flap is replaced.

### DETAILED DESCRIPTION OF THE INVENTION AND THE PREFERRED EMBODIMENTS

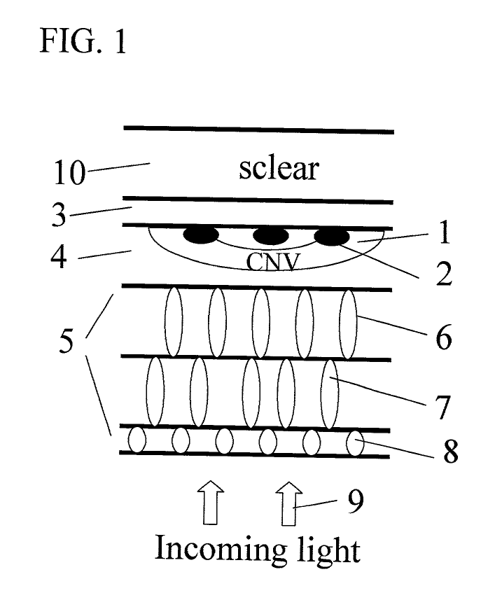

Referring to FIG. 1, The choroidal neovascularization (CNV) 1 is caused by the development of abnormal blood vessels (or drusen) 2 in the sub-retinal space either between the choriocapillaris 3 and the retinal pigment epithelum (RPE) 4 or between the RPE and neurosonsory retine 5 which consists of photoreceptor 6, bipolar cells 7 and optic nerve fibers 8. The incoming light is shown as 9 and the sclear layer is shown as 10. (From chapter 1, Physiology of the Eye, second ed. By I. Fatt and B. Weissman, Butterworth-Heinemann, MA, 1992). The presence of the abnormal vessels (or drusen), combined with the development of subretinal transudates and hemorrhage, ultimately results in irreparable damage to the overlying neurosensory retina (or phtoaceptor) 6 and permanent loss of vision. Formation of these drusen 2 is caused by the low flood flow and elevated pressure in the choriocapillaris 3 which in turn is caused by the rigidity of the sclera due to ageing.

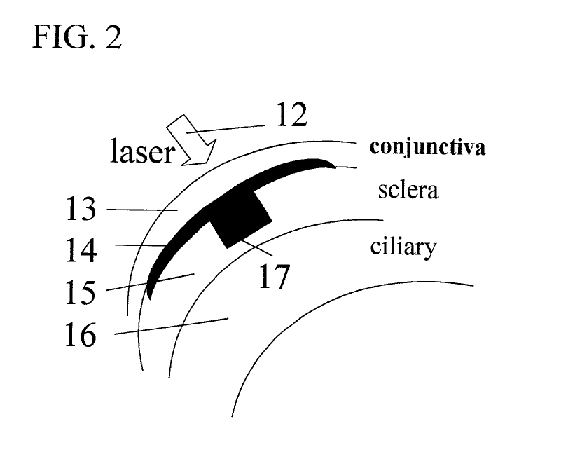

FIG. 2 shows various layers of eye tissue outside limbus: the conjunctiva 13, the sub-conjunctiva (or episclear) 14, the sclear 15, the diary body 16 and the sclera area 17 removed/ablated by a laser 12 The ablated area 17 having a depth about (50%-90%) of the sclera total scleral thickness is filled in by the sub-conjunctival tissue 14 after the conjunctiva flap is replaced. This filled-in sub-conjunctiva in the area 17 is much more elastic than the original scleral tissue 15, therefore the overall scleral layer surrounding the eye globe will become more elastic. The reduction the age-caused stiffness of the scleral tissue will lead the increase of flood flow and decrease of choriocapillary pressure and reduce the risk factors causing AMD or CNV.

Referring to FIG. 2, according to the present invention, the preferred embodiments of the basic surgical laser 12 for the prevention of AMD (or CNV) shall include: (a) infrared (IR) lasers having wavelengths range of about (1.4-3.2) microns including but not limited to solid state lasers of Er:glass, Ho:YAG, Er:YAG, Er:YSGG, infrared gas lasers, solid-state lasers converted by optical parametric oscillation (OPO); (b) ultraviolet (UV) lasers having wavelength range of about (190-360) nm, such as ArF (at 193 nm) and XeCl (at 308 nm) excimer lasers, nitrogen laser (at 337 nm) and solid-state lasers using harmonic generation from solid-state lasers of Nd:YAG, Nd:YLF and alexandrite lasers frequency conversions; (c) semiconductor diode lasers at about 980 nm, (1.3-1.55) microns, and (1.8-2.1) microns; (d) diode-pumped solid state lasers having wavelength range of about (190-355) nm and (2.7-3.2) microns such as diode-pumped Er:YSGG, Er:YAG, Nd:YAG and Er.glass; (e) short pulsed solid state laser at about (0.5-1.3) microns with pulse width less than 10 nanoseconds.

According to one aspect of the present invention, the preferable scanning laser energy per pulse on scleral surface is about (1-40) mJ in IR lasers and about (0.1-5.0) mJ in UV lasers and (0.001-0.1) mJ in ultra-short pulsed lasers. Focused spot size of about (0.1-2.0) mm in diameter on the scleral surface is proposed. The other preferred laser parameter of this invention is the laser repetition rate range of about (1-100) Hz which will provide reasonable surgical speed and minimum thermal effects. The focused beam may be scanned over the scleral surface to ablate various patterns either by a computer controlled scanner or manually held fiber coupler consists of a hand piece and a fiber tip.

The preferred patterns of this invention include a ring-spot having at least one ring with at least 3 spots in each ring, and a radial-pattern or a curved patterns having at least 3 radials or curves or any non-specific shapes as far as they are symmetrically in positions. The preferred area of the laser ablation is outside the limbus such that the corneal refractive power will not be affected by the procedure while the slceral is ablated. We also propose that the ablation pattern on the scleral surface may be generated either by an automatic scanning device or by manually scan the fiber tip by a surgeon who hold the hand piece or other scanning device suing refractive optics or rotating prisms. When a fiber is used, the fiber tip may be penetrate into the sclera layer without open the conjunctival layer and ablate the underlaying tissue or open the conjunctival layer before the laser ablating the sclera tissue. The fiber delivery unit may be operated in a contact-mode or non-contact mode to ablate the sclera tissue.

The ablation depth of the sclera ciliary tissue shall be about (60%-90%) of the total scleral thickness for safety reasons and optimal clinical outcomes. Any other non-specific patterns including curved lines, z-shape, t-shape lines around the area outside the limbus should be within the scope of this patent.

The preferred embodiment without opening the conjunctiva layer and inserting the fiber tip through the conjunctiva layer to ablate the sclera tissue underneath is a less invasive procedure than the one which opens the conjunctiva before the sclaral ablation. To do this procedure, the conjunctiva layer may be lifted to generate a “gap” for fiber tip to insert into this gap and ablate the desired patterns underneath. Additional advantages of this minimum invasive method is to avoid or minimize bleeding or infection. We note that most of the bleeding is due to cutting of the conjunctiva tissue rather than the laser ablation of the sciera tissue.

It is yet another preferred embodiment is to remove, by non-laser methods, portion of the sclera tissue which is filled in by sub-conjunctiva tissue to increase the flexibility of the scleral area. The preferred embodiment for these non-laser methods shall include, but not limited to, physical blades or knife, electromagnetic wave such as radio frequency wave, electrode device, bipolar device and plasma assisted electrode device. The electromagnetic wave generator is commercially available. However, the parameters of the device such as its frequency, pulse duration and repetition rate and the size of the electrode tip shall be selected for efficient cutting (or ablation) with minimum thermal damage to the tissue to be removed.

It is yet another preferred embodiment is to remove, by laser or non-laser methods, portion of the sclera tissue for the prevention, delaying or treatment of glaucoma by reducing the intraocular pressure (IOP).

While the invention has been shown and described with reference to the preferred embodiments thereof, it will be understood by those skilled in the art that the foregoing and other changes and variations in form and detail may be made therein without departing from the spirit, scope and teaching of the invention. Accordingly, threshold and apparatus, the ophthalmic applications herein disclosed are to be considered merely as illustrative and the invention is to be limited only as set forth in the claims.

I claim:

1. A surgery method of preventing age-related macular degeneration (AMD) by reducing the rigidity of the sclera of an eye, whereby a removal means is used to remove portion of the sclera tissue in a predetermined pattern to reduce the risk factors of AMD selected from the group consisting of low blood flow and high pressure in the choriocapillaris and formation of drusen in the suretinal space.

2. A surgical method as claimed in claim 1, wherein said removal means includes a laser having a preferable wavelength of about (0.15-3.2) microns.

3. A surgical method as claimed in claim 1, wherein said removal means includes a physical blades.

4. A surgical method as claimed in claim 1, wherein said removal means includes an electromagnetic wave at radio frequency, or electrode device, or bipolar device or plasma assisted electrode device.

5. A surgery method of preventing AMD in accordance with claim 1, in which said predetermined pattern includes patterns selected from the group consisting of radial lines, curved lines, ring-dot and non-specific patterns around the area of the eye outside the limbus.

6. A surgery method of preventing AMD in accordance with claim 1, in which said rigidity of the sclera is reduced by the filling effects of the sub-conjunctival tissue.

7 A surgical apparatus to remove a portion of the sclera tissue of an eye comprising
(a) a tissue removal means, and
(b) a control means of predetermined ablation pattern,
whereby AMD can be prevented by reducing the rigidity of the sclera.

8. A surgical apparatus as claimed in claim 7, wherein said removal means included a laser having a wavelength range of about (0.15-0.36) microns.

9. A surgical apparatus as claimed in claim 7, wherein said removal means included infrared laser having a wavelength range of about (0.8-3.2) microns.

10. A surgical apparatus as claimed in claim 8, wherein said laser is selected from the group consisting of ArF excimer laser, XeCl excimer laser, harmonic generation from Nd:YAG laser and Nd:YLF laser.

11. A surgical apparatus as claimed in claim 9, wherein said infrared laser is an optically pumped Erbium:YAG laser having a wavelength of about 2.9 microns

12. A surgical apparatus as claimed in claim 9, wherein said infrared laser is a solid-state short pulse laser having a wavelength range of about (1.0-1.3) microns and a pulse duration shorter than 10 nanoseconds.

13. A surgical apparatus as claimed in claim 9, wherein said infrared laser is a semiconductor diode laser having a wavelength range of about (0 8-2.1) microns

14. A surgical apparatus as claimed in claim 7, wherein said control means is selected from the group consisting of a scanning device having motorized reflection mirror, a refractive optics and optical device

15 A surgical apparatus as claimed in claim 7, wherein said control means includes the use of a fiber delivery unit to deliver said laser in a predetermined pattern onto a plurality of positions on the eye.

16. The apparatus of claim 15, wherein said fiber delivery unit is operated by a mode selected the group consisting of contact-mode and non-contact mode to ablate the sclera tissue.

17. A surgical apparatus as claimed in claim 15, wherein said fiber delivery unit is controlled by the surgeon to perform a predetermined patterns outside the limbus of the eye by manually moving said fiber delivery unit.

18. A surgical apparatus as claimed in claim 7, wherein said predetermined pattern is selected from the group consisting of radial lines, curved lines, ring-dot and non-specific pattern around the area outside the limbus.

19. A surgical apparatus as claimed in claim 7, wherein said removal means includes a physical blades.

20. A surgical apparatus as claimed in claim 7, wherein said removal means is selected from the group consisting of electromagnetic wave at radio frequency, electrode device, bipolar device oand plasma assisted electrode device.

21. A surgical apparatus to remove a portion of the sclera tissue of an eye comprising:
(c) a tissue ablation means, and
(d) a control means of predetermined ablation pattern,
whereby glaucoma can be prevented or treated by decreasing the intraocular pressure of the treated eye.

22. A surgical apparatus as claimed in claim 21, wherein said tissue ablation means includes a laser having a wavelength range of about (0.15-3.2) microns.

23. A surgical apparatus as claimed in claim 21, wherein said tissue ablation means is selected from the group consisting of a physical blades, an electromagnetic wave at radio frequency, oelectrode device, bipolar device and plasma assisted electrode device.

24. A surgical apparatus as claimed in claim 21, wherein said predetermined pattern is selected from the group consisting of radial lines, curved lines, ring-dot and non-specific pattern around the area outside the limbus.
